SEQUENCE LISTING (8 sequences)

SEQ ID NO: 1 - DNA, 20 bp, synthetic construct
  source 1..20: mol_type = other DNA; note = Upstream of GAPDH
gatgctggtgctgagtatgt

SEQ ID NO: 2 - DNA, 20 bp, synthetic construct
  source 1..20: mol_type = other DNA; note = Downstream of GAPDH
gcagaaggtgcagagatgat

SEQ ID NO: 3 - DNA, 18 bp, synthetic construct
  source 1..18: mol_type = other DNA; note = Upstream of SGPL1
attgtgtggagtgtcgtg

SEQ ID NO: 4 - DNA, 18 bp, synthetic construct
  source 1..18: mol_type = other DNA; note = Downstream of SGPL1
atcttccttggtcttgtt

SEQ ID NO: 5 - RNA, 22 nt, synthetic construct
  source 1..22: mol_type = other RNA; note = Upstream of miRNA sequence in interference group
aggcagtgtaattagctgattg

SEQ ID NO: 6 - RNA, 22 nt, synthetic construct
  source 1..22: mol_type = other RNA; note = Downstream of miRNA sequence in interference group
tttccgtcacattaatcgacta

SEQ ID NO: 7 - RNA, 22 nt, synthetic construct
  source 1..22: mol_type = other RNA; note = miRNA sequence in high expression group
caatcagctaattacactgcct

SEQ ID NO: 8 - RNA, 21 nt, synthetic construct
  source 1..21: mol_type = other RNA; note = miRNA sequence in blank control
cagtacttttgtgtagtacaa

(19) United States
(12) Patent Application Publication
(10) Pub. No.: US 2025/0163518 A1
(43) Pub. Date: May 22, 2025

(54) USE OF SGPL1 IN BREEDING OF SHANDONG BLACK CATTLE

(71) Applicant: Qingdao Agricultural University, Qingdao (CN)

(72) Inventors: Yajuan DONG, Qingdao (CN); Ruili LIU, Qingdao (CN); Wenqi ZHANG, Qingdao (CN); Xuejin BO, Qingdao (CN); Zhenpeng CHEN, Qingdao (CN)

(21) Appl. No.: 18/755,125

(22) Filed: Jun. 26, 2024

(30) Foreign Application Priority Data
Nov. 22, 2023 (CN) 202311563334.5

(51) Int. Cl. C12Q 1/6888

(52) U.S. Cl. CPC C12Q 1/6888; C12Q 2600/156; C12Q 2600/158

(57) ABSTRACT

Use of SGPL1 in regulating lipid metabolism of a Shandong Black cattle is provided, which belongs to the technical field of molecular biological breeding. The SGPL1 can be used in breeding of the Shandong Black cattle to screen an individual with a low SGPL1 level as a stud bull. Experiments have shown that the SGPL1 can regulate proliferation and growth of an adipose tissue of the Shandong Black cattle, thereby regulating a birth weight of the calf. The SGPL1 can also affect proliferation and growth of adipocytes of the Shandong Black cattle, thereby affecting growth of a fetal cattle. In breeding, the individual with a high SGPL1 level should be selected as a high-quality calf of the Shandong Black cattle. Therefore, a novel direction is provided for breeding a high-quality Blcattle beef cattle.

### CROSS REFERENCE TO RELATED APPLICATION

This patent application claims the benefit and priority of Chinese Patent Application No. 202311563334.5 filed with the China National Intellectual Property Administration on Nov. 22, 2023, the disclosure of which is incorporated by reference herein in its entirety as part of the present application.

### REFERENCE TO SEQUENCE LISTING

A computer readable XML file entitled “SEQUENCE LISTING”, that was created on Jun. 12, 2024, with a file size of about 9422 bytes, contains the sequence listing for this application, has been filed with this application, and is hereby incorporated by reference in its entirety.

### TECHNICAL FIELD

The present disclosure belongs to the technical field of molecular biological breeding, and specifically relates to use of an SGPL1 in regulating a beef quality of a Shandong Black cattle

### BACKGROUND

Carcass traits such as carcass weight, dressing rate, and meat-to-bone ratio are the main indicators reflecting the beef production performance of a beef cattle. As an important component of livestock and poultry meat products, adipose tissue is also an important factor affecting the meat quality. During breeding meat-producing animals, an intramuscular fat (IMF) content is the main indicator of meat quality. Therefore, studies have revealed that a genetic mechanism affecting the IMF content can pose certain guiding significance for increasing the meat production of livestock and poultry and improving the meat quality, thereby improving selective breeding standards and meat value in the beef industry.

Sphingolipids are ubiquitous structural components in mammalian cell membranes and participate in signal transduction and cell recognition pathways. Sphingolipid synthesis involves a series of tightly-regulated enzymatic steps that progress from non-sphingolipid precursors in the endoplasmic reticulum to higher-order complex sphingolipid biosynthesis in the Golgi apparatus. Despite the diversity of biosynthetic pathways, sphingolipid metabolism arguably starts from a common entry point and ends through a single degradation pathway.

Sphingosine 1 phosphate lyase 1 (SGPL1) is a key enzyme in the sphingolipid metabolism as well as the only intracellular enzyme that catabolizes sphingosine 1-phosphate (S1P). SGPL1 catalyzes the cleavage of S1P and other long-chain base phosphates (LCBPs) at positions C2 to C3 to generate ethyl ammonium phosphate and hexadecenol. The S1P is a biologically-active sphingolipid signaling molecule that participates in many important signal transduction processes such as cell growth, differentiation, aging, and programmed death. The SGPL1 affects the metabolic homeostasis of sphingolipids in the body by directly regulating S1P levels in vivo. Studies by BEKTAS et al. have shown that compared with normal mice, the levels of sphingolipid intermediate metabolites sphingosine, dihydrosphingosine, and ceramide are increased in the serum and liver of SGPL1-knockout mice, while the levels of phospholipids, TG, diacylglycerol, and cholesterol are also increased in non-sphingolipid metabolic pathways. This indicates that SGPL1 not only regulates sphingolipid metabolism, but also affects the metabolism of other lipids. Further studies have shown that the SGPL1 mutation causes the upregulation of both fat synthesis and decomposition and oxidation genes in adipose tissue, but mainly oxidation and decomposition. In addition, the SGPL1 promotes the proliferation and migration of lung adenocarcinoma cells, and promotes the upregulation of the lipolysis rate-limiting enzyme HSL and fatty acid oxygen key enzyme CPT1 as well as the expression of LPL and PPAR-γ. Taken together, it is shown that the SGPL1 plays an important role in lipid and fat metabolism. Overexpression of the SGPL1 can cause serum lipid metabolism disorders, enhanced fat oxidation and decomposition, and reduced fat content in animals. This mechanism has potential clinical significance and provides new research ideas and regulatory targets for regulating lipid metabolism.

Shandong Black cattle is the first novel beef cattle germplasm successfully bred through somatic cell nuclear transfer in China. Cross-breeding and molecular marker-assisted breeding techniques have overcome the shortcomings of Luxi Yellow and Bohai Black. The semen of Japanese Black is used to improve Luxi Yellow, such that its offspring can show a combination of excellent traits. The Shandong Black cattle has a tall body, well-developed muscles, obvious tendons, thin skin and bones, a strong constitution, and a well-proportioned structure. The beef from this breed appears tender and juicy, shows a desirable mouth feel, and is also called “snowflake beef” due to a typical marble pattern. Moreover, the beef of Shandong Black cattle is rich in protein with an amino acid composition closer to the demands of human body than that of other breeds, and can improve the disease resistance in vivo, nourish the heart muscle, and enhance immunity. Meanwhile, the beef is rich in iron, has an extremely low saturated fatty acid content in intermuscular fat and a high unsaturated fatty acid content, and exhibits a unique flavor. In 2015, this breed was recognized by experts as a novel breed that inherited the specialized beef breed characteristics of Japanese Black as well as an excellent breed of cattle for producing marbled beef. However, there are currently few studies on beef quality improvement and stud bull selection for this breed.

### SUMMARY

In order to solve the problems existing in the prior art, the present disclosure provides use of an SGPL1 in regulating lipids of a Shandong Black cattle, thereby promoting proliferation and growth of an adipose tissue to affect the birth weight of a calf.

To achieve the above objective, the present disclosure adopts the following technical solutions:

The present disclosure provides use of an SGPL1 in breeding or beef production of a Shandong Black cattle.

The use refers to detecting the following items using the SGPL1 as a target:
a messenger RNA expression level and a transcriptional or post-transcriptional modification level of an SGPL1 gene; or
mutation and a single nucleotide polymorphism (SNP) of the SGPL1 gene; or
an expression level and a post-translational modification level of an SGPL1 protein; or
a function, localization, and an activity of the SGPL1 protein.

In the present disclosure, the use refers to screening an individual with a high transcription level, a high expression level, or a high activity of the SGPL1 as a stud bull.

In the present disclosure, the use refers to retaining a calf after birth with a high transcription level, a high expression level, or a high activity of the SGPL1 to allow beef production.

The present disclosure may have the following advantages:

In the present disclosure, experiments prove that the expression of SGPL1 gene regulates the growth and invasion of preadipocytes and promotes fat synthesis, thereby affecting the beef production performance of a beef cattle. In breeding, individuals with a high SGPL1 content should be selected as indicators of the stud bull with a high-quality beef-producing performance, thus providing a novel direction for the breeding of Blcattle beef cattle.

### BRIEF DESCRIPTION OF THE DRAWINGS

FIGS. 1A-B shows the transcriptome data validation histogram (FIG. 1A) and the volcano plot of differential gene numbers (FIG. 1B);

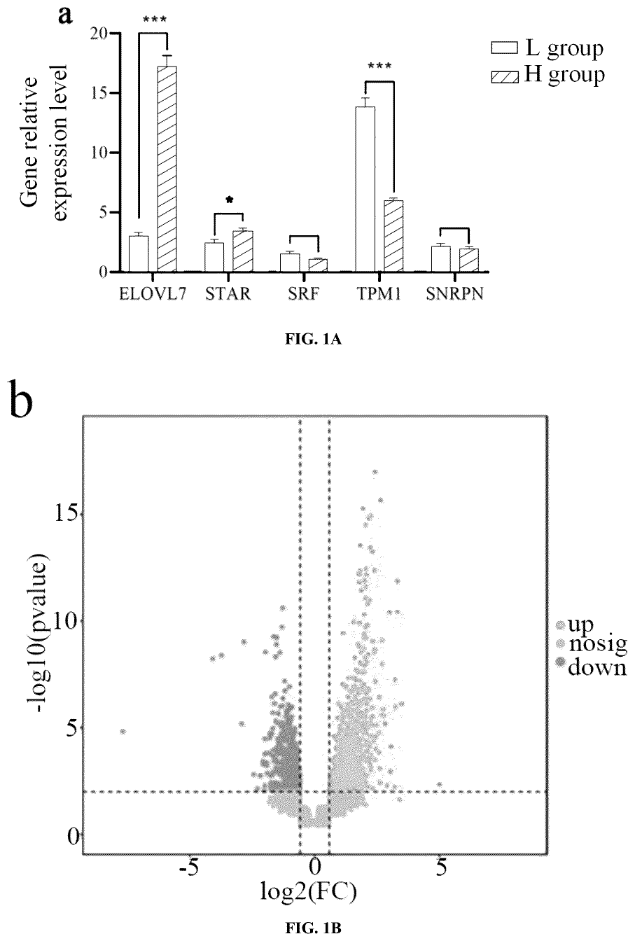

FIGS. 2A-B shows GO analysis (FIG. 2A) and KEGG enrichment analysis (FIG. 2B) of the differential genes;

FIG. 3 shows the correlation analysis between the SGPL1 and the carcass weight of a black cattle;

FIGS. 4A-B shows the expression levels of SGPL1 in different tissues (FIG. 4A) and different groups (FIG. 4B);

FIG. 5 shows an influence of expression of the SGPL1 gene on adipocyte migration;

FIG. 6 shows an influence of expression of the SGPL1 gene on adipocyte invasion;

FIG. 7 shows the expression levels of genes related to proliferation and differentiation of preadipocytes;

FIG. 8 shows the detection of lipid droplets by oil red O staining; and

FIG. 9 shows the glycerol contents in adipocytes of different treatment groups.

### DETAILED DESCRIPTION OF THE EMBODIMENTS

The following further describes the present disclosure with reference to the specific examples, but the present disclosure is not limited by the examples.

Example 1 Screening of Differential Genes Related to Calf Carcass Weight of Shandong Black Cattle

1. Calf phenotype and complete transcriptome library construction of Shandong Black cattle

Shandong Black cattle were selected from Shandong Zhaofu Animal Husbandry Co., Ltd. In the experiment, 8 healthy 18-month-old black cattle were selected and raised in the same gestation house with desirable lighting and ventilation, appropriate house temperature and humidity, and had consistent feeding and drinking water. Before slaughter, the phenotypic data such as carcass weight, leg circumference, chest circumference, body height, and daily weight gain data of the black cattle were measured. The significant difference indicators were shown in Table 1. According to the carcass weight of beef cattle, there were two groups: a low carcass weight group (L group) with 4 cattle and a high carcass weight group (H group) with 4 cattle. Their longissimus dorsi muscle, fat, heart, spleen, liver, and lung tissues were collected and immediately stored in liquid nitrogen, and then moved to an ultra-low temperature freezer for storage until use.

TABLE 1
Phenotypic data of Shandong Black cattle in groups H and L

| | High carcass | Low carcass |
|---|---|---|
| | weight group | weight group |
| Indicators, cm | (H group) | (L group) |
| Body height | 133.20 ± 0.800** | 125.20 ± 0.663 |
| Waist height | 136.50 ± 0.775** | 127.60 ± 0.187 |
| Body length | 165.50 ± 0.632*** | 155.50 ± 0.500 |
| Chest circumference | 200.20 ± 2.177 | 185.10 ± 2.216 |
| Waist circumference | 221.10 ± 1.778* | 205.40 ± 2.249 |
| Abdomen circumference | 246.40 ± 4.155 | 228.90 ± 1.806 |
| Cannon circumference | 20.70 ± 0.200 | 19.4 ± 0.187 |
| Buttock length | 54.60 ± 0.400 | 52.5 ± 0.500 |

Notes:
data were expressed as mean ± standard error,
*indicated significant difference (P < 0.05),
**indicated extremely significant difference (P < 0.01).

A total RNA from muscle tissue was extracted using Trizol reagent (Invitrogen). The extracted RNA sample with high integrity and purity was sent to Novogene (en.novogene.com) as samples for constructing sequence libraries, and a strand-specific cDNA library was constructed and paired-end transcriptome sequencing was conducted using a next-generation sequencer Illumina NovaSeq 6000. Trimmomatic (v0.36) software was used to filter out adapter sequences, primer sequences, and low-quality sequencing data, and FastQC software was used to evaluate the quality of the filtered sequencing data. A total of 44,090,358 clean reads were obtained, with an overall mapping rate of 95.62%, indicating that the sequencing data was correct and could be used for subsequent statistical analysis.

2. Screening of Differential Genes

2.1 Differential Expression Analysis

For experiments with biological replicates, DEseq was used for differential expression analysis; and for samples without biological replicates, DEGseq (DEGs) was used for differential expression analysis. By comparing the treatment group with the reference group and selecting genes with | log 2 Ratio|≥1 and q<0.05 as significant differential expression screening conditions, 580 differentially-expressed mRNAs were obtained, of which 238 were up-regulated and 342 were down-regulated genes.

2.2 GO and KEGG Enrichment Analysis

In order to identify the potential biological functions of DEGs genes, GO and KEGG enrichment analysis was conducted using the R package clusterProfiler. GO terms could be divided into three categories: biological process (BP), cellular composition (CC), and molecular function (MF). The threshold for significantly enriched GO Terms and KEGG pathways was set at FDR<0.05.

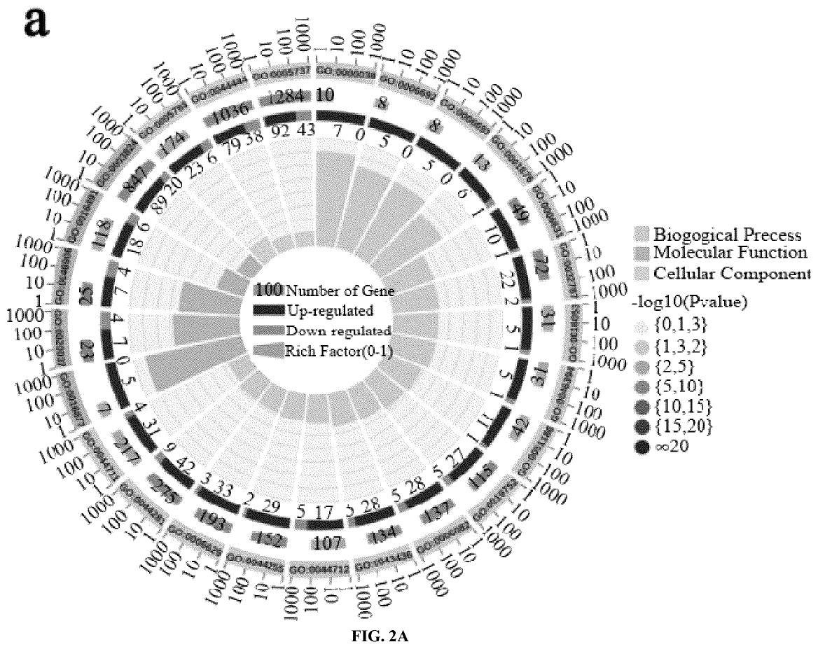

The results are shown in FIG. 2. The GO enrichment results show that DEGs genes are mainly enriched in molecular function items, including long-chain fatty acid CoA ligase activity, fatty acid ligase activity and other secondary items. There is also a small enrichment in cellular components (fatty acid-long chain alkane complexes, lipid particles). KEGG enrichment results show that DEGs genes are mainly enriched in energy metabolism-related processes such as the regulation of actin skeleton, tight junctions, and protein processing in the endoplasmic reticulum.

2.3 Weighted Gene Co-Expression Analysis (WGCNA)

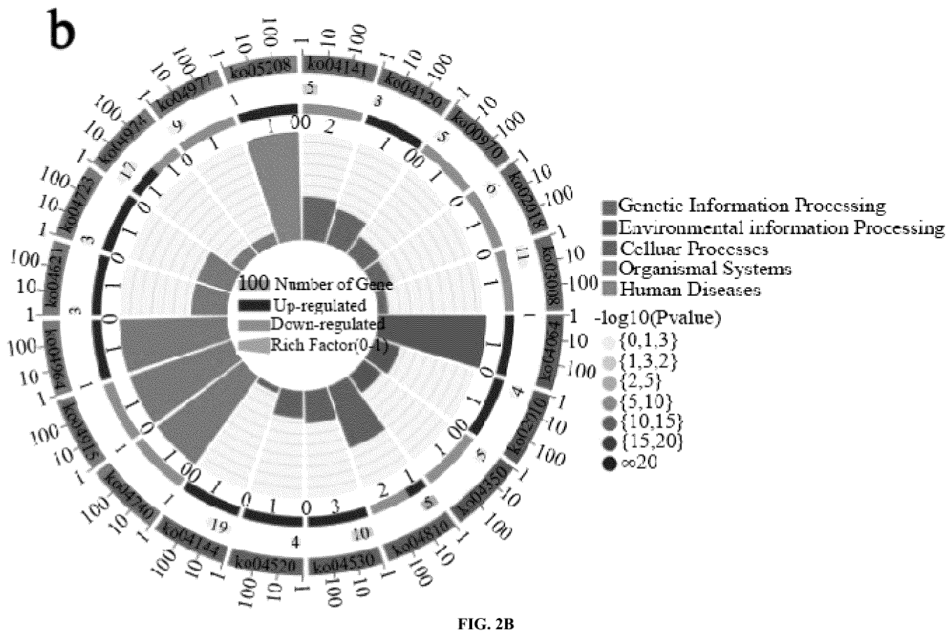

To elucidate the potential biological functions of differential genes in meat production performance, co-expression modules were identified from transcriptome data. A total of 5 co-expression modules were obtained and assigned 5 colors for easy visual differentiation (FIG. 3). In order to evaluate the correlation between each module and 11 meat quality traits, a correlation analysis was conducted between the modules and meat quality traits using a weighted gene co-expression analysis process, specifically including:

1) Input data preparation: the fpkm expression matrix was read to allow log 2(x+1) transformation; the matrix was transposed into the form of behavioral samples and genes.

2) Determining data quality and drawing a systematic clustering tree of samples: the data quality of samples and genes was checked, and low-quality data were removed; a systematic clustering tree of the sample was plotted, an existing significant outlier needed to be eliminated; a PCA plot could also be made to check the sample distribution.

3) Selecting the best threshold power: selection criteria: R²>0.8, slope≈−1, a power value was selected at the inflection point of the relationship between R² and power.

4) Construction of a weighted co-expression network (one-step method and step-by-step method) and identification of gene modules: gene modules and phenotypes were associated: gene modules and phenotypes were correlated: module and phenotype correlation heat map, module and phenotype correlation boxplot diagram, gene and module, and phenotype correlation scatter plot.

5) WGCNA standard heat map and module correlation display: WGCNA's standard heat map TOMPlot/Network heapmap plot depicted the topological overlap matrix (TOM) between all genes in the analysis. The darker the color, the greater the correlation was between genes; Eigengene-adjacency-heatmap displayed the correlation between gene modules. Here, the “black”, “pink”, and “royalblue” modules that were highly correlated with the phenotype were selected for GO analysis.

The results show that module 2 is significantly positively correlated with body weight, body height, and abdominal circumference separately (correlation>0.80, P<0.001) (FIG. 3). These results indicate that module 2 exhibited specific expression of meat-producing traits. When these mRNAs are ranked according to their responsiveness to body weight (effect size and statistical significance), the mRNA with the highest responsiveness to body weight is identified as SGPL1.

### Example 2 Expression Profile Identification of SGPL1

The placental tissues of the HW group and the LW group (4 animals in each group) were selected, while muscles, hearts, kidneys, livers, spleens, and adipose tissues of 2-month-old Shandong Black cattle (3 heads) were collected and stored in liquid nitrogen. The total RNA was extracted with Trizol reagent (Invitrogen), and a cDNA was obtained by reverse transcription. The cDNA was used as a template to conduct SYBR Green qPCR on an SGPL1 gene using glyceraldehyde-3-phosphate dehydrogenase (GAPDH) as a reference gene. A CT value of each sample was detected and averaged, and relative expression levels of the SGPL1 in different groups and tissues were calculated.

TABLE 2
qPCR primers for each gene

| Gene name | Primer | Sequence 5′-3′ |
|---|---|---|
| GAPDH | Upstream | GATGCTGGTGCTGAGTATGT |
| | | (SEQ ID NO: 1) |
| | Downstream | GCAGAAGGTGCAGAGATGAT |
| | | (SEQ ID NO: 2) |
| SGPL1 | Upstream | ATTGTGTGGAGTGTCGTG |
| | | (SEQ ID NO: 3) |
| | Downstream | ATCTTCCTTGGTCTTGTT |
| | | (SEQ ID NO: 4) |

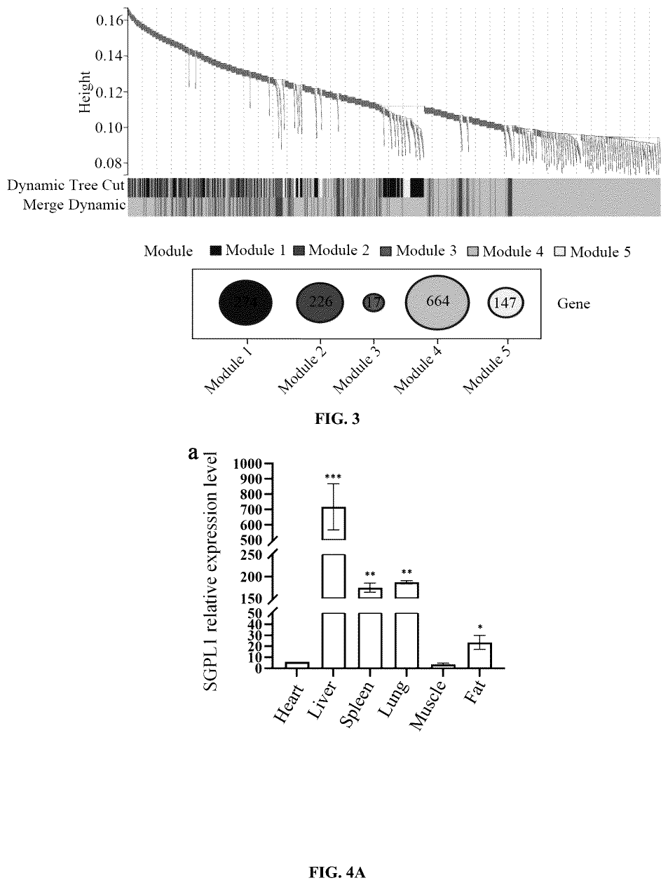

As shown in FIG. 4A, the results show that the mRNA has the highest expression level in the liver of Shandong Black cattle (P<0.01), followed by lung, spleen and fat, and the lowest expression level in muscle and heart.

### Example 3 Influence of SGPL1 Expression Level on Adipocytes in Shandong Black Cattle

1. Construction of Adipocytes With Differential Expression of SGPL1

Targeting miRNAs were selected based on the mRNA sequence of SGPL1 (GenBankNo.NM_001098053.1), and the sequence was shown in Table 3. Bovine primary cells were isolated and cultured from the hindlimb fat of newborn Shandong Black cattle calves. When the cells grew to 80% confluence, the cell suspension was a concentration-adjusted with complete medium and divided into 6-well plates, 2×10⁵ cells/well, 2 mL per well, cultured in a 37° C., 5% CO₂ incubator for 24 h, transfected with the miRNA of SGPL1 high expression group, the miRNA of SGPL1 interference group, and the miRNA of blank control (NC) for 48 h, then the medium was changed to a high-glucose DMEM medium with 2% horse serum to continue the culturing. Cells were collected starting at 48 h for subsequent detection. The results of the fluorescence quantitative test show (FIG. 4B) that compared with the blank group, the expression of SGPL1 in the interference group is significantly reduced (P<0.05), and the expression of SGPL1 in the high-expression group is extremely significantly increased (P<0.01). This indicates that 48 h after preadipocytes are transfected with miRNA, the miRNA targetedly regulated the expression of SGPL1, and there are significant differences among the groups. The transfected cells can be used for subsequent verification experiments.

TABLE 3
Construction of miRNAs in adipocytes with differential SGPL1 expression

| Group | miRNA sequence |
|---|---|
| Interference group | 5′-3′: AGGCAGUGUAAUUAGCUGAUUG (SEQ ID NO: 5) |
| | 3′-5′: AUCAGCUAAUUACACUGCCUUU (SEQ ID NO: 6) |
| High expression group | 5′-3′: CAAUCAGCUAAUUACACUGCCU (SEQ ID NO: 7) |
| Blank control | 5′-3′: CAGUACUUUUGUGUAGUACAA (SEQ ID NO: 8) |

2. Influence of SGPL1 on Adipocyte Migration and Invasion in Shandong Black Cattle

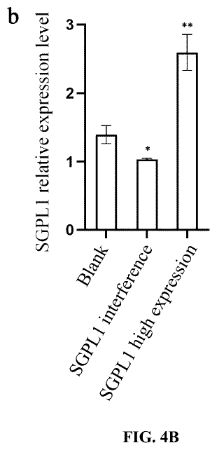

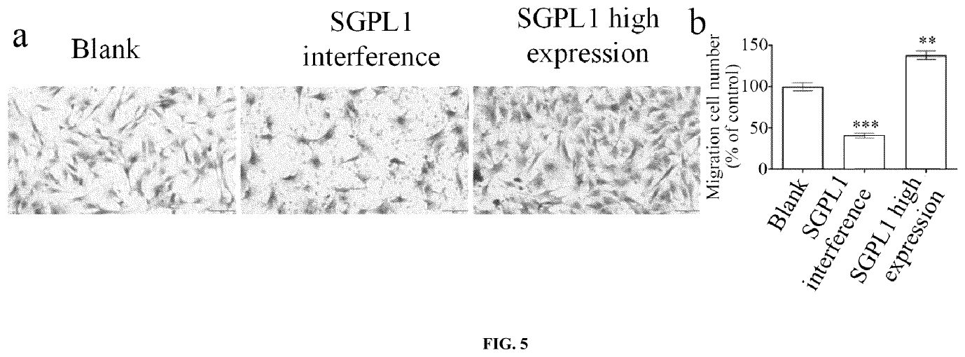

48 h after the preadipocytes were transfected with the miRNA sequence, the cells were collected for Transwell migration and invasion testing: the cells were digested, the medium was removed by centrifugation after terminating the digestion, washed 2 times with PBS, resuspended in serum-free medium, and the cell density was adjusted to 1.5×10⁵ cells/mL. 200 μL of cell suspension was added to the Transwell well, 600 μL of medium containing 10% FBS was added to the lower chamber of the 24-well plate, and cultured at 37° C. for 24 h. The Transwell well was taken out, the medium in the well was discarded, and wash 2 times with PBS; for the invasion test, Matrigel needed to be diluted 5 times with serum-free medium, and 50 μL of the medium was added to each Transwell well. Cells were fixated with 4% PFA for 10 min and washed 2 times with PBS. The cells were stained with crystal violet for 10 min, washed 2 times with PBS, and the cells in the upper chamber were wiped off with cotton swabs. After observing the migrating and invasive cells under a microscope, 3 fields of view were randomly selected to take pictures and counted. Cell migration (FIG. 5) and invasion (FIG. 6) results showed that high expression level of SGPL1 significantly increased the migration and invasion of preadipocytes, while the opposite was true for the interference group.

3. Influence of SGPL1 on Adipocyte Differentiation and Proliferation in Shandong Black Cattle

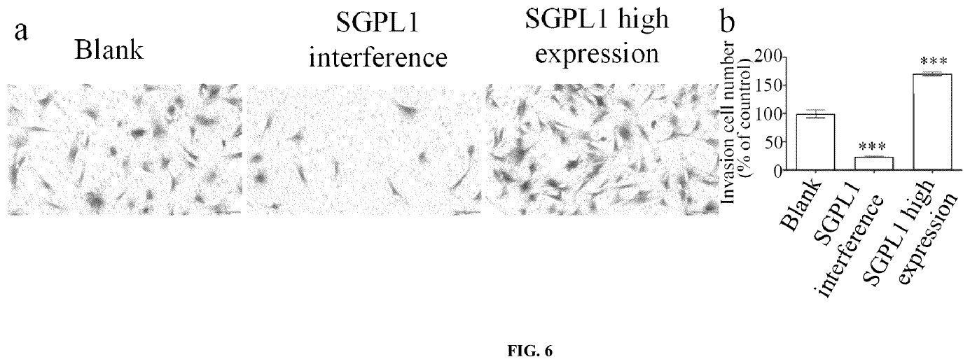

qRT-PCR was conducted to detect the expression levels of proliferation marker genes ((CND1, CDK4, and PCNA) and differentiation marker genes (PPARG, CEBPA, and FABP4), and the results were shown in FIG. 7. The results showed that high expression level of SGPL1 significantly increased the expression levels of differentiation marker genes (P<0.01), and also significantly increased the expression levels of proliferation marker genes (CDK4, PCNA) (P<0.01), while the opposite was true for the interfering with SGPL1. It was seen that the SGPL1 promoted the proliferation and differentiation of preadipocytes at the same time, but mainly promoted differentiation.

### Example 4 Influence of SGPL1 Gene Expression on Fat Synthesis and Glycerol Content

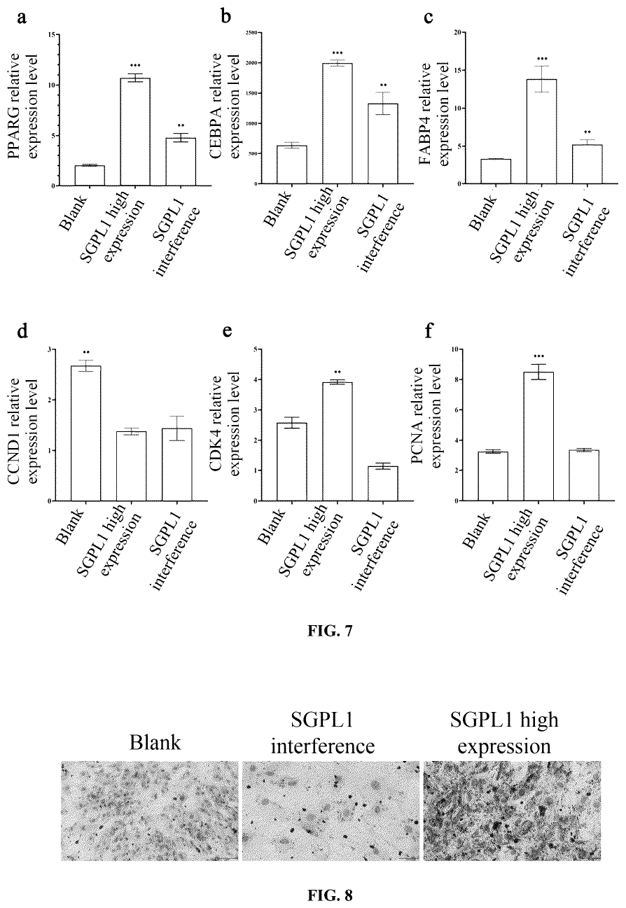

The primary cells of Shandong Black cattle were differentiated by preadipocyte induction for 48 h, and the medium was changed to a high-glucose DMEM medium with 2% horse serum to continue the culturing. After adipogenic induction and differentiation, the cells were collected for oil red O staining. The complete medium was removed from the 24-well plate and washed 3 times with 1×PBS. 500 μL of 4% paraformaldehyde solution was added to each well and fixate at room temperature for 30 min. A working solution was prepared according to oil red O storage solution: distilled water=3:2. After mixing, centrifugation was conducted at 250×g for 4 min and a supernatant was collected. The fixative was removed, the cells were washed 3 times with 1×PBS, 500 μL of oil red O dye working solution was added to each well, and stained at room temperature for 30 min. The oil red O dye working solution was removed, the cells were washed 3 times with 1×PBS, 500 μL hematoxylin was added to each well, and stained at room temperature for 2 min. 500 μL of 1×PBS was added to each well, and the culture plate was placed under a microscope to observe the adipogenic staining effect. Oil red O staining results (FIG. 8) show that compared with the blank group, the number of lipid droplets in the SGPL1 high-expression group is significantly increased, and the number of lipid droplets in the SGPL1 interference group is significantly reduced, indicating that SGPL1 promoted fat synthesis.

After adipogenic induction and differentiation, the cells were collected for triglyceride content detection test. 4-5 million adipocytes were collected into a centrifuge tube, centrifuged and the supernatant was discarded, added with 1 mL of reagent I (n-heptane and isopropyl alcohol mixed at a volume ratio of 1:1), and ultrasonicated for 1 min (intensity 20%, ultrasonicated for 2 s, stopped for 1 s), centrifuged at 8,000 g, 4° C. for 10 min, and the supernatant was collected for testing. A spectrophotometer/plate reader was preheated for 30 min, adjusted to wavelength of 420 nm, and zeroed with distilled water. A water bath was preheated to 65° C. The reagent I was added and mixed thoroughly, then reagent II was added (triglyceride (TG) content detection kit), shaken vigorously for 30 s, allowed to stand for 3 min, then shaken vigorously for 30 s, repeated 3 times, and allowed to stand at room temperature for a certain period of time. After layering, 30 μL of the upper solution was collected and added into a new EP tube. After cooling, 200 μL of the upper solution was pipetted into a micro glass cuvette/96-well plate and the absorbance was measured at 420 nm, recorded as A_{blank}, A_{standard}, and Aₜₑₛₜ, the blank tube and standard tube needed to be tested 1-2 times. The triglyceride (TG) content in cells was calculated according to the following formula:

$$TG content ( mg / {10}^{4} cell ) = {C}_{standard} \times ( {A}_{t e s t} - {A}_{blank} ) \div ( {A}_{standard} - {A}_{blank} ) \div cell density ; \\ or , \\ TG content ( mg / {10}^{4} cell ) = ( {A}_{t e s t} - {A}_{blank} ) \div ( {A}_{standard} - {A}_{blank} ) \div cell density ; \\ {C}_{standard} : 1 m g / mL ; cell density : {10}^{4} cell s / mL .$$

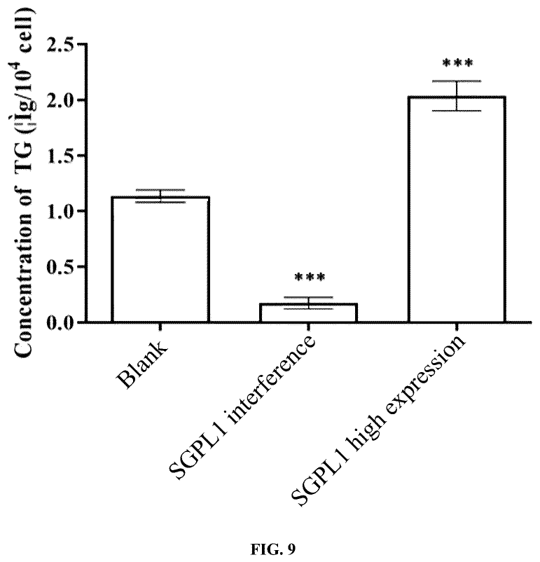

The results of TG content detection test are shown in FIG. 9. The results show that compared with the blank group, the TG content in adipocytes of the SGPL1 high-expression group is significantly increased, while that of the interference group is significantly decreased. The results show that SGPL1 can promote glycerol synthesis in adipocytes.

What is claimed is:

1. A screening method in breeding or beef production of a Shandong Black cattle, wherein the method comprises detecting the following items using the SGPL1 as a target:
a messenger RNA expression level and a transcriptional or post-transcriptional modification level of an SGPL1 gene; or
mutation and a single nucleotide polymorphism (SNP) of the SGPL1 gene; or
an expression level and a post-translational modification level of an SGPL1 protein; or
a function, localization, and an activity of the SGPL1 protein.

2. The method according to claim 1, wherein the method further comprises screening an individual with a high transcription level, a high expression level, or a high activity of the SGPL1 as a stud bull.

3. The method according to claim 1, wherein the method further comprises retaining a calf with a high transcription level, a high expression level, or a high activity of the SGPL1 to allow beef production.
